(19) United States
(12) Patent Application Publication
(10) Pub. No.: US 2010/0096585 A1
(43) Pub. Date: Apr. 22, 2010

(54) Process for Preparing 2,6-Dichloro-4-(Trifluoromethyl)Phenylhydrazine Using Mixtures of Dichloro-Fluoro-Trifluoromethylbenzenes

(75) Inventors: Thomas Zierke, Bohl-Iggelheim (DE); Michael Rack, Eppelheim (DE)

Correspondence Address: BRINKS, HOFER, GILSON & LIONE, P.O. BOX 1340, MORRISVILLE, NC, 27560, US

(73) Assignee: BASF SE, Ladwigshafen (DE)

(21) Appl. No.: 12/528,888

(22) PCT Filed: Feb. 27, 2008
(86) PCT No.: PCT/EP2008/052341

§ 371 Date: Aug. 27, 2009

(30) Foreign Application Priority Data
Mar. 16, 2007 (EP) 07104345.9

(51) Int. Cl. C09K 3/00; C07C 241/02

(52) U.S. Cl. 252/182.12; 564/314

(57) ABSTRACT

This invention relates to a process for preparing 2,6-dichloro-4-(trifluoromethyl)phenylhydrazine of the formula (I) wherein a mixture comprising 1,3-dichloro-2-fluoro-5-trifluoromethylbenzene and 1,2-dichloro-3-fluoro-5-trifluoromethylbenzene is reacted with a hydrazine source selected from hydrazine, hydrazine hydrate or acid addition salts of hydrazine, optionally in the presence of at least one organic solvent.

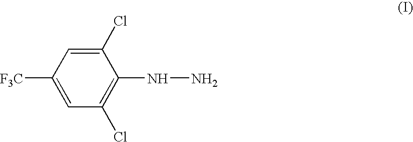

The present invention relates to a process for preparing 2,6-dichloro-4-(trifluoromethyl)phenylhydrazine of the formula I

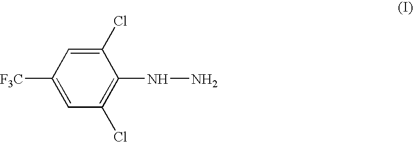

wherein mixtures of dichloro-fluoro-trifluoromethylbenzenes are used as starting materials.

2,6-dichloro-4-(trifluoromethyl)phenylhydrazine of the formula I (synonym name: 1-[2,6-dichloro-4-(trifluoromethyl) phenyl]hydrazine) is an important intermediate product for the preparation of various pesticides (see, for example, WO 00/59862, EP-A 0 187 285, WO 00/46210, EP-A 096645, EP-A 0954144 and EP-A 0952145).

A number of methods are known for preparing 2,6-dichloro-4-(trifluoromethyl) phenylhydrazine of the formula I.

EP-A 0 187 285 describes the preparation of 2,6-dichloro-4-(trifluoromethyl) phenylhydrazine by the reaction of 3,4,5-trichlorotrifluoromethyl-benzene with hydrazine hydrate in pyridine at a temperature of from 115 to 120° C. (see preparation example 1).

This procedure, however, must be conducted at relatively high temperatures and suffers from limited selectivity. Moreover, the reaction mixture obtained from the conversion of 3,4,5-trichlorotrifluoromethyl-benzene requires a tedious and difficult separation of the desired end product from its isomers due to the close proximity of their melting points.

It is therefore an object of the present invention to provide an improved method for preparing 2,6-dichloro-4-(trifluoromethyl)phenylhydrazine of the formula I, in particular to find procedures which can be performed at moderate temperatures and allows for a higher selectivity and also an easier separation and isolation of the desired end product from the reaction mixture.

This object is achieved by a process for preparing 2,6-dichloro-4-(trifluoromethyl)phenylhydrazine of the formula I, wherein a mixture comprising 1,3-dichloro-2-fluoro-5-trifluoromethylbenzene of the formula II

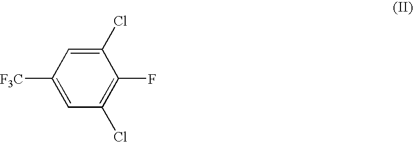

and 1,2-dichloro-3-fluoro-5-trifluoromethylbenzene of the formula III

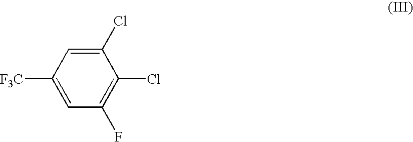

(hereinafter also simply referred to as the “mixture”)
is reacted with a hydrazine source selected from hydrazine, hydrazine hydrate and acid addition salts of hydrazine, optionally in the presence of at least one organic solvent (A), to form 2,6-dichloro-4-(trifluoromethyl)phenylhydrazine of the formula I.

It has surprisingly been found that, by using the mixture as defined herein as starting material, 2,6-dichloro-4-(trifluoromethyl)phenylhydrazine of the formula I can be obtained under milder conditions compared to prior art processes and with a selective conversion of the 1,3-dichloro-2-fluoro-5-trifluoromethylbenzene of the formula II present in the mixture and an easier separation and isolation of the desired end product from the reaction mixture.

In general, the hydrazine source is used in an at least equimolar amount or in a slight excess, relative to the molar amount of 1,3-dichloro-2-fluoro-5-trifluoromethylbenzene of the formula II present in the mixture. Preference is given to using 1 to 6 moles, in particular from 1 to 4 moles, and more preferably from 1 to 3 moles of the hydrazine source, relative to 1 mole of 1,3-dichloro-2-fluoro-5-trifluoromethylbenzene of the formula II present in the mixture.

In a preferred embodiment, the mixture is reacted with hydrazine hydrate. The amount of hydrazine hydrate is generally from 1 to 6 moles, in particular from 1 to 4 moles and more preferably from 1 to 3 moles, relative to 1 mole of 1,3-dichloro-2-fluoro-5-trifluoromethylbenzene of the formula II present in the mixture.

The term “acid addition salts of hydrazine” refers to hydrazine salts formed from strong acids such as mineral acids (e.g. hydrazine sulfate and hydrazine hydrochloride).

The molar ratio of 1,3-dichloro-2-fluoro-5-trifluoromethylbenzene of the formula II to 1,2-dichloro-3-fluoro-5-trifluoromethylbenzene of the formula III in the mixture is usually from 3:1 to 9:1, in particular from 3.2:1 to 9:1, and more preferably from 3.3:1 to 9:1.

In a preferred embodiment, the mixture comprises from 65 to 98% by weight, in particular 70 to 95% by weight, and more preferably 70 to 90% by weight of 1,3-dichloro-2-fluoro-5-trifluoromethylbenzene of the formula II and from 2 to 35% by weight, in particular 5 to 30% by weight, and more preferably 10 to 30% by weight of 1,2-dichloro-3-fluoro-5-trifluoromethylbenzene of the formula III, all weight percentages being based on the total weight of the mixture.

The process according to the invention may in principle be carried out in bulk, but preferably in the presence of at least one organic solvent (A).

Suitable organic solvents (A) are practically all inert organic solvents including cyclic or aliphatic ethers such as dimethoxyethan, diethoxyethan, bis(2-methoxyethyl) ether (diglyme), triethyleneglycoldimethyl ether (triglyme), dibutyl ether, methyl tert-butyl ether, tetrahydrofuran, 2-methyltetrahydrofuran, dioxane and the like; aromatic hydrocarbons such as toluene, xylenes (ortho-xylene, meta-xylene and para-xylene), ethylbenzene, mesitylene, chlorobenzene, dichlorobenzenes, anisole and the like; alcohols such as methanol, ethanol, n-propanol, isopropanol, n-butanol and the like; tertiary C₁-₄ alkylamines such as triethylamine, tributylamine, diisoproylethylamine and the like; heterocyclic aromatic compounds such as pyridine, 2-methylpyridine, 3-methylpyridine, 5-ethyl-2-methylpyridine, 2,4,6-trimethylpyridine (collidine), lutidines (2,6-dimethylpyridine, 2,4-dimethylpyridine and 3,5-dimethylpyridine), 4-dimethylaminopyridine and the like; and any mixture of the aforementioned solvents.

Preferred organic solvents (A) are cyclic ethers (in particular those as defined hereinabove), alcohols (in particular those as defined hereinabove), aromatic hydrocarbons (in particular those as defined hereinabove) and heterocyclic aromatic compounds (in particular those as defined hereinabove), and any mixture thereof. More preferably, the organic solvent (A) is selected from cyclic ethers (in particular from those as defined hereinabove) and aromatic hydrocarbons (in particular from those as defined hereinabove), and any mixture thereof.

Thus, a broad variety of organic solvents (A) can surprisingly be utilized in the process according to this invention including non-polar solvents, weakly polar solvents, polar protic solvents and polar aprotic solvents.

In a preferred embodiment, non-polar or weakly polar organic solvents having a dielectric constant of not more than 12, preferably not more than 8 at a temperature of 25° C. are used as organic solvent (A) in the process according to this invention. Such non-polar or weakly polar organic solvents can be selected from among a variety of organic solvents known to a skilled person, in particular from those listed hereinabove. Specific examples of organic solvents (A) fulfilling the above requirements include aromatic hydrocarbons, in particular toluene (having a dielectric constant of 2.38 at 25° C.), and cyclic ethers, in particular tetrahydrofuran (having a dielectric constant of 7.58 at 25° C.).

Preferred organic solvents (A) are aromatic hydrocarbons, in particular those as listed hereinabove and any mixture thereof. Toluene is most preferred among the aromatic hydrocarbons.

Preference is also given to the use of heterocyclic aromatic compounds organic solvent (A), in particular those as listed hereinabove and any mixture thereof, and most preferably pyridine.

The most preferred organic solvents (A) are cyclic ethers, in particular cyclic ethers having from 4 to 8 carbon atoms, and more preferably tetrahydrofuran.

The organic solvent (A) is generally used in an amount of from 1 to 20 moles, in particular from 2 to 15 moles, and more preferably from 3 to 10 moles, relative to 1 mole of 1,3-dichloro-2-fluoro-5-trifluoromethylbenzene of the formula II present in the mixture.

The process according to the invention may be conducted at a temperature up to the boiling point of the reaction mixture. Advantageously, the process can be carried out at an unexpectedly low temperature, such as below 60° C. The preferred temperature range is from 0° C. to 60° C., more preferably 10° C. to 55° C., yet more preferably 15° C. to 50° C., even more preferably 15° C. to 45° C., even still more preferably 20° C. to 40° C. and most preferably 20° C. to 30° C.

The reaction of the mixture with the hydrazine source can be carried out under reduced pressure, normal pressure (i.e. atmospheric pressure) or increased pressure. Preference is given to carrying out the reaction in the region of atmospheric pressure.

The reaction time can be varied in a wide range and depends on a variety of factors, such as, for example, the reaction temperature, the organic solvent (A), the hydrazine source and the amount thereof. The reaction time required for the reaction is generally in the range from 1 to 120 hours, preferably 1 to 24 hours.

The mixture comprising 1,3-dichloro-2-fluoro-5-trifluoromethylbenzene of the formula II and 1,2-dichloro-3-fluoro-5-trifluoromethylbenzene of the formula III and the hydrazine source may be contacted together in any suitable manner. Frequently, it is advantageous that the mixture is initially charged into a reaction vessel, optionally together with the organic solvent desired, and the hydrazine source is then added to the resulting mixture.

The reaction mixture can be worked up and 2,6-dichloro-4-(trifluoromethyl)phenylhydrazine of the formula I can be isolated therefrom by using known methods, such as washing, extraction, precipitation, crystallization and distillation.

If desired, 2,6-dichloro-4-(trifluoromethyl)phenylhydrazine of the formula I can be purified after its isolation by using techniques that are known in the art, for example by distillation, recrystallization and the like.

The conversion of 1,3-dichloro-2-fluoro-5-trifluoromethylbenzene of the formula II present in the mixture usually exceeds 50%, in particular 70%, more preferably 80% and even more preferably 90%.

The conversion is usually measured by evaluation of area-% of signals in the gas chromatography assay of a sample taken from the reaction solution (hereinafter also referred to as “GC area-%”). For the purposes of this invention, conversion is defined as the ratio of the difference of the GC area-% of 1,3-dichloro-2-fluoro-5-trifluoromethylbenzene of the formula II assayed in the initial reaction mixture minus the GC area-% of not converted 1,3-dichloro-2-fluoro-5-trifluoromethylbenzene of the formula II assayed in the reaction mixture after completion of the reaction against the GC area-% of 1,3-dichloro-2-fluoro-5-trifluoromethylbenzene of the formula II assayed in the initial reaction mixture, with said ratio being multiplied by 100 to obtain the percent conversion.

1,3-dichloro-2-fluoro-5-trifluoromethylbenzene of the formula II and 1,2-dichloro-3-fluoro-5-trifluoromethylbenzene of the formula III contained in the mixture are known compounds and may be prepared by known methods, such as those described in EP-A 0 034 402, U.S. Pat. No. 4,388,472, U.S. Pat. No. 4,590,315, Journal of Fluorine Chemistry, 30 (1985), pp. 251-258, EP-A 0 187 023 (see Example 6) or in an analogous manner.

In a preferred embodiment, the mixture is obtained by reacting 1,2,3-trichloro-5-trifluoromethylbenzene of formula IV

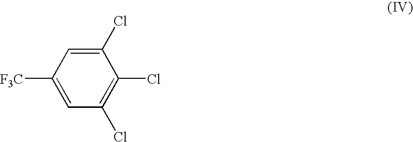

with a fluorinating agent, optionally in the presence of at least one organic solvent (B).

1,2,3-trichloro-5-trifluoromethylbenzene of formula IV is a known compound and can be prepared by known methods (see e.g. DE-OS 2 644 641 and U.S. Pat. No. 2,654,789).

The reaction of 1,2,3-trichloro-5-trifluoromethylbenzene of formula IV with the fluorinating agent is herein also referred to as the “fluorine-chlorine exchange”.

Examples of suitable fluorinating agents are alkali metal fluorides (e.g. potassium fluoride, sodium fluoride and caesium fluoride), alkali earth metal fluorides (e.g. calcium fluoride), and mixtures thereof. Preference is given to using alkali metal fluorides, in particular potassium fluoride. The alkali metal fluoride and/or alkali earth metal fluoride may be used in a spray-dried or crystalline form.

In another embodiment, the present invention relates to a process for the preparation of a mixture comprising 1,3-dichloro-2-fluoro-5-trifluoromethylbenzene of the formula II and 1,2-dichloro-3-fluoro-5-trifluoromethylbenzene of the formula III, wherein 1,2,3-trichloro-5-trifluoromethylbenzene of formula IV is reacted with a fluorinating agent, optionally in the presence of at least one organic solvent (B), said fluorinating agent being selected from alkali metal fluorides, alkali earth metal fluorides, and mixtures thereof. Preferred alkali metal fluorides or preferred alkali earth metal fluorides are the same as those listed above. It is even more preferred to use alkali metal fluorides, in particular potassium fluoride. The alkali metal fluoride and/or alkali earth metal fluoride can likewise be used in a spray-dried or crystalline form.

It is preferred to carry out the fluorine-chlorine exchange using a slight excess of the fluorinating agent. The amount of the fluorinating agent is generally from 1.05 to 2.0 moles, in particular from 1.1 to 1.5 moles and more preferably from 1.15 to 1.3 moles, relative to 1 mole of 1,2,3-trichloro-5-trifluoromethylbenzene of formula IV.

The reaction of 1,2,3-trichloro-5-trifluoromethylbenzene of formula IV with the fluorinating agent may in principle be carried out in bulk, but preferably in the presence of at least one organic solvent (B), and more preferably in an inert organic solvent (B) under water-free conditions. Suitable organic solvents (B) that may be employed include, for example, aromatic hydrocarbons such as toluene, ortho-xylene, meta-xylene, para-xylene and the like; halogenated aromatic hydrocarbons such as chlorobenzene; dialkyl sulfoxides such as dimethylsulfoxide, diethylsulfoxide, dipropylsulfoxide, dioctylsulfoxide and the like; alkylene ureas such as N,N′-dimethylethylene urea (DMEU), N,N′-dimethyl propylene urea (DMPU) and the like; carboxylic acid amides including N,N-dialkyl formamides such as N,N-dimethylformamide (DMF), N,N-diethylformamide and the like, and N,N-dialkyl acetamides such as N,N-dimethylacetamide (DMA); dialkyl sulfones such as dimethyl sulfone, diethyl sulfone and the like; diaryl sulfones such as diphenyl sulfone; N-alkyl 2-pyrrolidones such as N-methyl 2-pyrrolidone (NMP); tetrahydrothiophen-1,1-dioxide (sulfolane); and any mixture of the aforementioned solvents. Particularly preferred are N,N′-dimethylethylene urea (DMEU), N,N′-dimethyl propylene urea (DMPU), N-methyl 2-pyrrolidone (NMP), tetrahydrothiophen-1,1-dioxide (sulfolane), and any mixture thereof.

Generally, the fluorine-chlorine exchange can be conducted over a period of time in the range of 3 to 16 hours.

The fluorine-chlorine exchange is generally conducted at a temperature of from 90° C. to 315° C. In the preferred embodiment where alkali metal fluorides and/or alkali earth metal fluorides are employed as the fluorinating agent, the temperature range is from 100° C. to 300° C., preferably from 170° C. to 230° C.

In another embodiment of the process of this invention, the fluorine-chlorine exchange is preferably carried out in the presence of a phase transfer catalyst.

Phase-transfer catalysts which have hitherto been used for the halogen-fluorine exchange reaction (also known as the halex reaction) are, for example, quaternary alkylammonium or alkylphosphonium salts (U.S. Pat. No. 4,287,374), pyridinium salts (WO 87/04194), crown ethers or tetraphenylphosphonium salts (J. H. Clark et al., Tetrahedron Letters 28, 1987, pages 111 to 114), guanidinium salts, aminophosphonium salts and polyaminophosphazenium salts (see, for example, U.S. Pat. No. 5,824,827, WO 03/101926, EP-A 1 070 723, EP-A 1 070 724, EP-A 1 266 904 and US 2006/0241300).

Examples of phase transfer catalysts suitable for the purpose of this invention include quaternary ammonium salts, quaternary phosphonium salts, guanidinium salts, pyridinium salts, crown ethers, polyglycols and mixtures thereof.

Also, one or more compounds of the following formulae (Va), (Vb) and (Vc) may be used

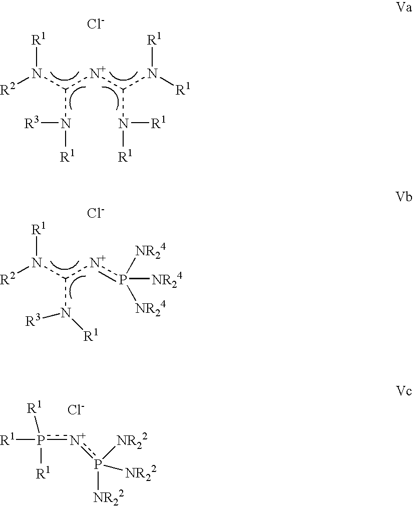

wherein, in the formulae Va and Vb, R¹ is C₁-₄ alkyl, R² and R³ collectively represent —CH₂-CH₂— or —CH₂-CH₂—CH₂— and R⁴ is C₁-₄ alkyl and, in the formula Vc, R¹ and R² are both C₁-₄ alkyl.

The term “C₁-C₄ alkyl”, as used hereinabove, refers to straight or branched aliphatic alkyl groups having from 1 to 4 carbon atoms, e.g. methyl, ethyl, propyl, isopropyl, n-butyl, sec-butyl and tert-butyl.

Concrete examples for quaternary ammonium salts are benzyl tributyl ammonium bromide, benzyl tributyl ammonium chloride, benzyl triethyl ammonium bromide, benzyl triethyl ammonium chloride, benzyl trimethyl ammonium chloride, cetyl trimethyl ammonium bromide, didecyl dimethyl ammonium chloride, dimethyl distearyl ammonium bisulfate, dimethyl distearyl ammonium methosulfate, dodecyl trimethyl ammonium bromide, dodecyl trimethyl ammonium chloride, methyl tributyl ammonium chloride, methyl tributyl ammonium hydrogen sulfate, methyl tricaprylyl ammonium chloride, methyl trioctyl ammonium chloride, myristyl trimethyl ammonium bromide, phenyl trimethyl ammonium chloride, tetrabutyl ammonium borohydride, tetrabutyl ammonium bromide, tetrabutyl ammonium chloride, tetrabutyl ammonium fluoride, tetrabutyl ammonium hydrogen sulfate, tetrabutyl ammonium hydroxide, tetrabutyl ammonium iodide, tetrabutyl ammonium perchlorate, tetraethyl ammonium bromide, tetraethyl ammonium chloride, tetraethyl ammonium hydroxide, tetrahexyl ammonium bromide, tetrahexyl ammonium iodide, tetramethyl ammonium bromide, tetramethyl ammonium chloride, tetramethyl ammonium fluoride, tetramethyl ammonium hydroxide, tetramethyl ammonium iodide, tetraoctyl ammonium bromide, tetrapropyl ammonium bromide, tetrapropyl ammonium chloride, tetrapropyl ammonium hydroxide, tributyl methyl ammonium chloride, triethyl benzyl ammonium chloride, and any mixture thereof.

Suitable guanidinium salts are, for example, hexa-C₁-C₆-alkyl guanidinium chloride, hexa-C₁-C₆-alkyl guanidinium bromide and any mixture thereof.

Specific examples of the quaternary phosphonium salts include benzyltriphenylphosphonium bromide, benzyltriphenylphosphonium chloride, butyltriphenylphosphonium bromide, butyltriphenylphosphonium chloride, ethyltriphenylphosphonium acetate, ethyltriphenylphosphonium bromide, ethyltriphenylphosphonium iodide, methyltriphenylphosphonium bromide, tetrabutylphosphonium bromide, tetraphenylphosphonium bromide, tetrakisdiethylaminophosphonium bromide, and any mixture thereof.

Concrete examples of pyridinium salts are cetyl pyridinium bromide, cetyl pyridinium chloride, and any mixture thereof.

Examples of crown ethers are 18-crown-6, dibenzo-18-crown-6 (e.g. Aliplex DB186®), and any mixture thereof.

Specific examples of polygycols include glycol diethers of the formula (VI)

CH₃(OCH₂CH₂)ₙOCH₃ (VI)

wherein n represents an integer of 1 to 50, in particular monoethylene glycol dimethyl ether (monoglyme), diethylene glycol dimethyl ether (diglyme), triethylene glycol dimethyl ether (triglyme), tetraethylene glycol dimethyl ether (tetraglyme), a glycol diether of the formula VI wherein n is 4 to 5 (e.g. Polyglycol DME 200®, Clariant), a glycol diether of the formula VI wherein n is 3 to 8 (e.g. Polyglycol DME 250®, Clariant), a glycol diether of the formula VI wherein n is 6 to 16 (e.g. Polyglycol DME 500®, Clariant), a glycol diether of the formula VI wherein n is 22 (e.g. Polyglycol DME 1000®, Clariant), and a glycol diether of the formula VI wherein n is 44 (e.g. Polyglycol DME 2000®, Clariant), dipropylene glycol dimethyl ether, diethylene glycol dibutyl ether (butyl diglyme), polyethylene glycol dibutyl ether, in particular a polyethylene glycol dibutyl ether having a molecular weight of 300 (e.g. Polyglycol BB 300®, Clariant), and any mixture thereof.

In a preferred embodiment, the phase transfer catalyst is selected from quaternary ammonium salts and quaternary phosphonium salts, preferably from quaternary phosphonium salts, more preferably from quaternary phosphonium bromides and is in particular tetraphenylphosphonium bromide.

If not commercially available, the aforementioned phase transfer catalysts can be prepared by procedures well known to those skilled in the art, e.g. such as by procedures described in U.S. Pat. No. 4,287,374, WO 87/04194, J. H. Clark et al., Tetrahedron Letters 28, 1987, pages 111 to 114, U.S. Pat. No. 5,824,827, WO 03/101926, EP-A 1 070 723, EP-A 1 070 724, EP-A 1 266 904 and US 2006/0241300, or in an analogous manner.

The amount of the phase transfer catalyst is generally from 0.01 to 0.02 moles, in particular from 0.01 to 0.1 moles and more preferably from 0.01 to 0.05 moles, relative to 1 mole of 1,2,3-trichloro-5-trifluoromethylbenzene of formula IV.

Advantageously, the fluorine-chlorine exchange is carried out in the presence of a reduction inhibitor, in particular when N,N-dimethylformamide (DMF) and/or N-methyl 2-pyrrolidone (NMP) are used as organic solvent (B). The reduction inhibitor is used in an understoichiometric amount, relative to 1,2,3-trichloro-5-trifluoromethylbenzene of formula IV. Suitable reduction inhibitors are, for example, 1,3-dinitrobenzene, 1-chloro-3-nitrobenzene, 4-chloro nitrobenzene, and any mixture thereof.

Preferably, the reaction mixture is worked up after the fluorine-chlorine exchange, and the mixture can be isolated therefrom by using conventional methods, such as washing, extraction and distillation. If desired, the mixture can be purified after its isolation by using techniques that are known in the art, for example by distillation, recrystallization and the like. As the fluorination products are liquids, the preferred purification technique is distillation. In a preferred embodiment, the resulting fluorination products are distilled off during the reaction. The removal of the fluorination products by distillation is preferably carried out under reduced pressure (vacuum distillation).

The reaction mixture may be dried directly by distillation of the organic solvent or by aceotropic distillation of a cosolvent. Preferably, aromatic hydrocarbons and/or halogenated aromatic hydrocarbons are used as cosolvents. Toluene, ortho-xylene, meta-xylene, para-xylene, chlorobenzene or any mixture thereof are preferred, with toluene being the most preferred.

A preferred embodiment of the invention relates to a process for preparing 2,6-dichloro-4-(trifluoromethyl)phenylhydrazine of the formula I comprising the steps of
a) reacting 1,2,3-trichloro-5-trifluoromethylbenzene of formula IV with a fluorinating agent as defined herein, optionally in the presence of at least one organic solvent (B) as defined herein, to obtain a mixture comprising 1,3-dichloro-2-fluoro-5-trifluoromethylbenzene of the formula II and 1,2-dichloro-3-fluoro-5-trifluoromethylbenzene of the formula III, and
b) reacting the mixture obtained from step (a) with a hydrazine source as defined herein, optionally in the presence of at least one organic solvent (A) as defined herein, to obtain 2,6-dichloro-4-(trifluoromethyl)phenylhydrazine of the formula I.

The steps (a) and (b) as defined hereinabove may be performed separately or in a one-pot procedure (i.e. without isolating the mixture obtained from step (a)).

Combinations of preferred embodiments with other preferred embodiments are within the scope of the present invention.

The process according to the invention has a number of advantages over the procedures hitherto used for the preparation of 2,6-dichloro-4-(trifluoromethyl) phenylhydrazine. In particular it has been shown that, by using the mixture as defined herein as starting material, the desired end product can be obtained under milder conditions compared to prior art processes and with a selective conversion of the 1,3-dichloro-2-fluoro-5-trifluoromethylbenzene of the formula II present in the mixture. The desired end product can be easily separated from the non-converted 1,2-dichloro-3-fluoro-5-trifluoromethylbenzene of the formula III. Moreover, the process of this invention makes it possible to use cheaply to produce technical grade 1,3-dichloro-2-fluoro-5-trifluoromethylbenzene of the formula II. Specifically, it is not necessary to use 1,3-dichloro-2-fluoro-5-trifluoromethylbenzene of a high purity with respect to the isomeric 1,2-dichloro-3-fluoro-5-trifluoromethylbenzene of the formula III, which may be difficult to separate from 1,3-dichloro-2-fluoro-5-trifluoromethylbenzene. Moreover, high conversions are achievable in a wide variety of solvents under mild reaction conditions. Furthermore, the use of cyclic ethers such as tetrahydrofuran and the use of a lower excess of hydrazine offer advantages compared to the prior art. This saves raw material costs and reduces also the efforts for waste disposal. In summary, the process of the present invention provides a more economic and industrially more feasible route to 2,6-dichloro-4-(trifluoromethyl)phenylhydrazine of the formula I.

The following Examples are illustrative of the process of this invention, but are not intended to be limiting thereof. The invention is further illustrated by the following Comparative Examples (not of the invention).

### EXAMPLE 1

### Preparation of a Mixture Comprising 1,3-dichloro-2-fluoro-5-trifluoromethylbenzene of the Formula II and 1,2-dichloro-3-fluoro-5-trifluoromethylbenzene of the Formula III

23 g (0.396 mol) KF, 12.8 g (0.03 mol) PPh₄Br, 91.2 g sulfolane and 152 ml toluene were mixed in a 500 ml reactor. Toluene was distilled off under reduced pressure (140° C., 60mbar; aceotropic removal of water). After cooling to 100° C., 76 g (0.305 mol) 1,2,3-trichloro-5-trifluoromethylbenzene were added and the resulting mixture was heated at 190° C. for 15 h under reduced pressure (100 mbar). The mixture of 1,3-dichloro-2-fluoro-5-trifluoromethylbenzene and 1,2-dichloro-3-fluoro-5-trifluoromethylbenzene was distilled off simultaneously via a column. Two distillation fractions were obtained, which contained 31% GC area-% of the product mixture, 1% GC area-% of difluoro compounds and 6.6% GC area-% of the educt 1,2,3-trichloro-5-trifluoromethylbenzene. The identity of the mixture was determined by GC/MS spectrometry and ¹⁹F-NMR spectroscopy.

### COMPARATIVE EXAMPLE 1

### Reaction of 1,2,3-trichloro-5-trifluoromethylbenzene (3,4,5-trichlorobenzotrifluoride) with tetraphenylphosphonium Hydrogen difluoride (tetraphenylphosphonium bifluoride)

1.12 g (0.0029 mol) of tetraphenylphosphonium hydrogen difluoride were added to 8.08 g (0.03 mol) of 1,2,3-trichloro-5-trifluoromethylbenzene and the resulting mixture was heated under reflux for 2 hours. The reaction mixture was allowed to cool and solved in water. The products were extracted with methyl tert-butylether. The conversion was determined by gas-chromatographic analysis. 0.15 GC area-% of 1,3-dichloro-2-fluoro-5-trifluoromethylbenzene of the formula II, 0.04 GC area-% of 1,2-dichloro-3-fluoro-5-trifluoromethylbenzene of the formula III, and 91.06% GC area-% of the educt 1,2,3-trichloro-5-trifluoromethylbenzene were obtained.

### COMPARATIVE EXAMPLE 2

### Reaction of 1,2,3-trichloro-5-trifluoromethylbenzene (3,4,5-trichlorobenzotrifluoride) with tetraphenylphosphonium hydrogen difluoride (tetraphenylphosphonium bifluoride) Employing a 1:1 Stoichiometry of the Reactants

1.12 g (0.0029 mol) of tetraphenylphosphonium hydrogen difluoride were added to 0.75 g (0.003 mol) of 1,2,3-trichloro-5-trifluoromethylbenzene and the resulting mixture was heated under reflux for 2 hours. The reaction mixture was allowed to cool and solved in water. The products were extracted with methyl tert-butylether. The conversion was determined by gas-chromatographic analysis. 14.2 GC area-% of 1,3-dichloro-2-fluoro-5-trifluoromethylbenzene of the formula II, 4.2 GC area-% of 1,2-dichloro-3-fluoro-5-trifluoromethylbenzene of the formula III, and 44.6 GC area-% of the educt 1,2,3-trichloro-5-trifluoromethylbenzene were obtained.

### EXAMPLE 2

### Preparation of 2,6-dichloro-4-(trifluoromethyl) phenylhydrazine of the Formula I

7 g of the mixture as obtained in Example 1 containing 73.3 wt-% 1,3-dichloro-2-fluoro-5-trifluoromethylbenzene (22 mmol) of the formula II and 21.5 wt-% of 1,2-dichloro-3-fluoro-5-trifluoromethylbenzene (6 mmol) of the formula III were dissolved in 15 g of tetrahydrofuran (208 mmol). To this solution were added 3.6 g (72 mmole) of hydrazine hydrate (100%). The resulting mixture was stirred at 25° C. for 24 hours. Thereafter, an organic layer of 21.8 g was separated, which contained the product 2,6-dichloro-4-(trifluoromethyl) phenylhydrazine as a 23.3 wt-% solution in tetrahydrofuran, meaning that a yield of 94.1% based on accessible 1,3-dichloro-2-fluoro-5-trifluoromethylbenzene was obtained. The organic layer contained in addition 0.5 wt-% of 2,3-dichloro-5-trifluoromethyl) phenylhydrazine, meaning that 7% of the accessible 1,2-dichloro-3-fluoro-5-trifluoromethylbenzene has been converted to the isomeric phenylhydrazine. The identity of the products was deduced from the GC assay on the basis of comparison samples.

1-18. (canceled)

19. A process for preparing 2,6-dichloro-4-(trifluoromethyl)phenylhydrazine of the formula I 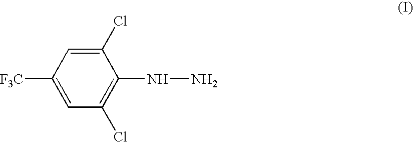
wherein a mixture comprising 1,3-dichloro-2-fluoro-5-trifluoromethylbenzene of the formula II 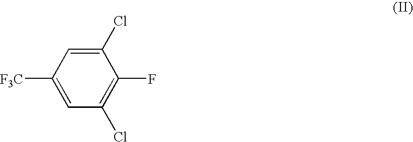
and 1,2-dichloro-3-fluoro-5-trifluoromethylbenzene of the formula III 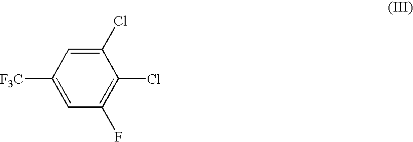
is reacted with a hydrazine source selected from the group consisting of hydrazine, hydrazine hydrate and acid addition salts of hydrazine, optionally in the presence of at least one organic solvent (A), to form 2,6-dichloro-4-(trifluoromethyl)phenylhydrazine of the formula I.

20. The process of claim 19, wherein said reaction of the mixture with the hydrazine source is carried out in the presence of at least one organic solvent (A).

21. The process of claim 20, wherein said organic solvent (A) is one or more cyclic ethers.

22. The process of claim 21, wherein said one or more cyclic ethers is tetrahydrofuran.

23. The process of claim 20, wherein said reaction is carried out at a temperature in the range of from 15° C. to 45° C.

24. The process of claim 19, wherein said hydrazine source is hydrazine hydrate.

25. The process of claim 24, wherein said hydrazine hydrate is used in an amount of 1 to 6 moles, relative to 1 mole of 1,3-dichloro-2-fluoro-5-trifluoromethylbenzene of the formula II present in said mixture.

26. The process of claim 19, wherein the molar ratio of 1,3-dichloro-2-fluoro-5-trifluoromethylbenzene of the formula II to 1,2 dichloro-3-fluoro-5-trifluoromethylbenzene of the formula III in the mixture is from 3:1 to 9:1.

27. The process of claim 19, wherein said mixture is obtained by reacting 1,2,3-trichloro-5-trifluoromethylbenzene of formula IV 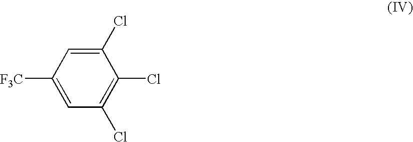
with a fluorinating agent, optionally in the presence of at least one organic solvent (B).

28. The process of claim 27, wherein said fluorinating agent is an alkali metal fluoride.

29. The process of claim 27, wherein said reaction of 1,2,3-trichloro-5-trifluoromethylbenzene of formula IV with the fluorinating agent is carried out in the presence of at least one organic solvent (B).

30. The process of claim 29, wherein said organic solvent (B) is tetrahydrothiophen-1,1-dioxide.

31. The process of claim 27, wherein said reaction of 1,2,3-trichloro-5-trifluoromethylbenzene of formula IV with the fluorinating agent is carried out in the presence of a phase transfer catalyst.

32. The process of claim 31, wherein said phase transfer catalyst is selected from quaternary phosphonium salts.

33. A process for the preparation of a mixture comprising 1,3-dichloro-2-fluoro-5-trifluoromethylbenzene of the formula II and 1,2-dichloro-3-fluoro-5-trifluoromethylbenzene of the formula III, wherein 1,2,3-trichloro-5-trifluoromethylbenzene of formula IV is reacted with a fluorinating agent, optionally in the presence of at least one organic solvent (B), said fluorinating agent being selected from alkali metal fluorides, alkali earth metal fluorides, and mixtures thereof.

34. The process of claim 33, wherein said fluorinating agent is an alkali metal fluoride.

35. The process of claim 34, wherein said alkali metal fluoride is potassium fluoride.
